(19) United States
(12) Patent Application Publication
(10) Pub. No.: US 2025/0129040 A1
(43) Pub. Date: Apr. 24, 2025

(54) METHODS FOR THE PREPARATION OF ETHYL 3-BROMO-1-(3-CHLOROPYRIDIN-2-YL)-4,5-DIHYDRO-1H-PYRAZOLE-5-CARBOXYLATE

(71) Applicants: FMC CORPORATION, Philadelphia, PA (US); FMC IP TECHNOLOGY GMBH, Neuhausen (CH)

(72) Inventors: Minghai LEI, Shanghai (CN); Fengdong YU, Shanghai (CN); Miao WANG, Shanghai (CN); Zongren HAN, Shanghai (CN)

(21) Appl. No.: 18/834,773

(22) PCT Filed: Jan. 30, 2023
(86) PCT No.: PCT/US2023/011827

§ 371 Date: Jul. 31, 2024

Related U.S. Application Data
(60) Provisional application No. 63/304,772, filed on Jan. 31, 2022.

(51) Int. Cl. C07D 401/04

(52) U.S. Cl. CPC C07D 401/04

(57) ABSTRACT

Described herein are methods of synthesizing ethyl 3-bromo-1-(3-chloropyridin-2-yl)-4,5-dihydro-1H-pyrazole-5-carboxylate.

### CROSS-REFERENCE TO RELATED APPLICATION

This application claims the benefit of U.S. Provisional Application No. 63/304,772 filed Jan. 31, 2022.

### FIELD OF INVENTION

This disclosure is directed to methods of synthesizing ethyl 3-bromo-1-(3-chloropyridin-2-yl)-4,5-dihydro-1H-pyrazole-5-carboxylate. Compounds prepared by the methods disclosed herein are particularly useful for preparation of certain anthranilamide compounds that are of interest as insecticides, such as, for example, the insecticides chlorantraniliprole and cyantraniliprole.

### BACKGROUND

The present disclosure provides novel methods useful for preparing ethyl 3-bromo-1-(3-chloropyridin-2-yl)-4,5-dihydro-1H-pyrazole-5-carboxylate and derivatives thereof. Methods to prepare ethyl 3-bromo-1-(3-chloropyridin-2-yl)-4,5-dihydro-1H-pyrazole-5-carboxylate are known in the art, such as those described in WO 2004/087689. However, these known methods are costly and wasteful.

Compared to conventional processes, the benefits of the methods of the present disclosure are numerous and include improved reduced waste, more flexibility in bromination reagent selection, decreased usage of bromination agent, increased yield, and reduced cost.

### BRIEF DESCRIPTION

In one aspect, provided herein is a method of preparing a compound of Formula (II)

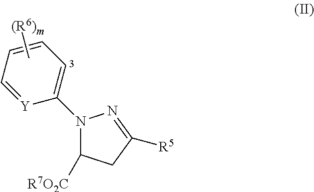

wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;
Y is N or CR⁸;
R⁸ is H or R⁶; and
m is 0, 1, 2, or 3, with the proviso that when X is CH then m is at least 1, the method comprising:
I) forming a mixture comprising
A) a compound of Formula (I)

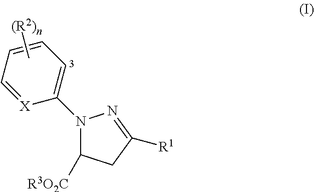

wherein R¹ is a sulfonate;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) a solvent; and
C) optionally a bromide;
II) introducing a bromination agent to the mixture;
III) introducing a strong acid to the mixture; and
IV) optionally adding a base to the mixture.

### DETAILED DESCRIPTION OF THE DISCLOSURE

Described herein are methods of synthesizing ethyl 3-bromo-1-(3-chloropyridin-2-yl)-4,5-dihydro-1H-pyrazole-5-carboxylate.

The embodiments of this disclosure include:

Embodiment 1. A method of preparing a compound of Formula (II)

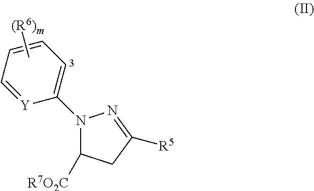

wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;
Y is N or CR⁸;
R⁸ is H or R⁶; and
m is 0, 1, 2, or 3, with the proviso that when X is CH then m is at least 1, the method comprising:
I) forming a mixture comprising
A) a compound of Formula (I)

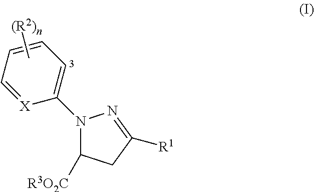

wherein R¹ is a sulfonate;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) a solvent; and
C) optionally a bromide;
II) introducing a bromination agent to the mixture;
III) introducing a strong acid to the mixture; and
IV) optionally adding a base to the mixture.

Embodiment 2. The method of embodiment 1, wherein m is 1, 2, or 3.

Embodiment 3. The method of any of embodiments 1-2, wherein R⁵ is Cl or Br.

Embodiment 4. The method of any of embodiments 1-3, wherein R⁶ is independently Cl or Br.

Embodiment 5. The method of any of embodiments 1-4, wherein one R⁶ is at the 3-position.

Embodiment 6. The method of any of embodiments 1-5, wherein R⁷ is C₁-C₄ alkyl.

Embodiment 7. The method of any of embodiments 1-6, wherein Y is N.

Embodiment 8. The method of any of embodiments 1-7, wherein the compound of Formula (II) is ethyl 3-bromo-1-(3-chloropyridin-2-yl)-4,5-dihydro-1H-pyrazole-5-carboxylate, having the following structure:

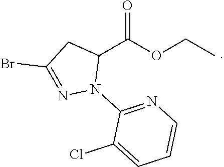 [SMILES: CCOC(=O)C1CC(Br)=NN1c1ncccc1Cl]

Embodiment 9. The method of any of embodiments 1-8, wherein n is 1, 2, or 3.

Embodiment 10. The method of any of embodiments 1-9, wherein R¹ is selected from methanesulfonate, benzenesulfonate, and p-toluenesulfonate.

Embodiment 11. The method of any of embodiments 1-10, wherein R² is independently Cl or Br.

Embodiment 12. The method of any of embodiments 1-11, wherein one R² is at the 3-position.

Embodiment 13. The method of any of embodiments 1-12, wherein R³ is C₁-C₄ alkyl.

Embodiment 14. The method of any of embodiments 1-13, wherein X is N.

Embodiment 15. The method of any of embodiments 1-14, wherein the compound of Formula (I) is ethyl 1-(3-chloropyridin-2-yl)-3-((phenylsulfonyl)oxy)-4,5-dihydro-1H-pyrazole-5-carboxylate, having the following structure:

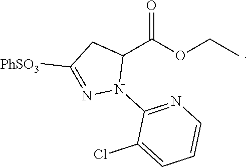

Embodiment 16. The method of any of embodiments 1-15, wherein the solvent is selected from dibromomethane, dichloromethane, acetic acid, ethyl acetate, acetonitrile, dichloroethane, dibromoethane, and combinations thereof.

Embodiment 17. The method of any of embodiments 1-16, wherein the solvent comprises acetic acid in an amount in a range of from about 0.1 eq. to about 10 eq., preferably about 0.5 eq. to about 6 eq.

Embodiment 18. The method of any of embodiments 1-17, wherein the bromide is selected from phosphorus oxybromide, phosphorus pentabromide, phosphorus tribromide, dibromo trialkyl phosphine and dibromo diphenyl phosphine, and combinations thereof.

Embodiment 19. The method of any of embodiments 1-18, wherein the bromination agent is selected from HBr, Br₂, and combinations thereof.

Embodiment 20. The method of any of embodiments 1-19, wherein the bromination agent is HBr.

Embodiment 21. The method of any of embodiments 1-20, wherein the strong acid is selected from sulfuric acid, hydrogen bromide, acids stronger than hydrogen bromide, and combinations thereof.

Embodiment 22. The method of any of embodiments 1-21, wherein the strong acid is sulfuric acid.

Embodiment 23. The method of any of embodiments 1-22, wherein the base is selected from sodium hydroxide, potassium hydroxide, sodium carbonate, sodium bicarbonate, potassium carbonate, potassium bicarbonate and combinations thereof.

Embodiment 24. The method of any of embodiments 1-23, wherein the base is sodium hydroxide.

Embodiment 25. The method of any of embodiments 1-24, wherein the method step of adding a base to the mixture comprises adding a base to the mixture through dropwise addition.

Embodiment 26. The method of any of embodiments 1-25, wherein at least one method step further comprises stirring the mixture.

Embodiment 27. The method of any of embodiments 1-26, wherein at least one method step further comprises cooling the mixture to a temperature in a range of from about 0° C. to about 5° C.

Embodiment 28. The method of any of embodiments 1-27, wherein at least one method step further comprises heating the mixture to a temperature in a range of from about 8° C. to about 12° C.

In one aspect, ethyl 3-bromo-1-(3-chloropyridin-2-yl)-4,5-dihydro-1H-pyrazole-5-carboxylate is prepared according to a method represented by Scheme 1.

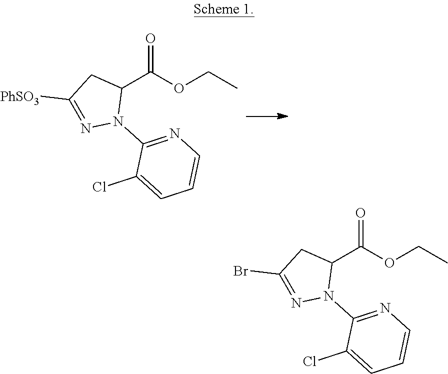

In one aspect, a compound of Formula II is prepared according to a method represented by Scheme 2. The R groups, X, Y, n, and m are as defined anywhere in this disclosure.

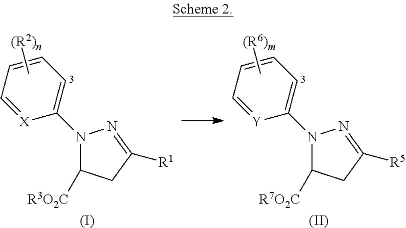

This aspect includes forming a mixture comprising a compound of Formula I, a solvent, and optionally a bromide, introducing a bromination agent to the mixture, introducing a strong acid to the mixture, and optionally adding a base to the mixture.

In some embodiments, the solvent is selected from dibromomethane, dichloromethane, acetic acid, ethyl acetate, acetonitrile, dichloroethane, dibromoethane, and combinations thereof.

Generally, any suitable amount of acetic acid may be used in the solvent. In many embodiments, the amount of acetic acid is relatively low to reduce usage of acetic acid and waste. Amounts of acetic acid that are too low will not be conducive to the reaction but relatively high amounts of acetic acid will produce more waste. In some embodiments, the solvent comprises acetic acid in an amount in a range of from about 0.1 eq. to about 10 eq. In some embodiments, the solvent comprises acetic acid in an amount in a range of from about 0.5 eq. to about 6 eq. In some embodiments, the solvent comprises acetic acid in an amount in a range of from about 0.7 eq. to about 3 eq. In some embodiments, the solvent comprises acetic acid in an amount in a range of from about 0.9 eq. to about 1.5 eq. In some embodiments, the solvent comprises acetic acid in an amount of about 1.0 eq.

In some embodiments, the bromide is selected from phosphorus oxybromide, phosphorus pentabromide, phosphorus tribromide, dibromo trialkyl phosphine and dibromo diphenyl phosphine, and combinations thereof. In some embodiments, the bromide reduces moisture content.

In some embodiments, the bromination agent is selected from HBr, Br₂, and combinations thereof. In some embodiments, the bromination agent is HBr.

Generally, any suitable amount of the bromination agent may be used. In many embodiments, the bromination agent is particularly useful for reducing waste.

In some embodiments, the bromination agent is present in an amount in a range of from about 0.1 eq. to about 10 eq. In some embodiments, the bromination agent is present in an amount in a range of from about 0.5 eq. to about 6 eq. In some embodiments, the bromination agent is present in an amount in a range of from about 0.7 eq. to about 3 eq. In some embodiments, the bromination agent is present in an amount in a range of from about 0.9 eq. to about 1.5 eq. In some embodiments, the bromination agent is present in an amount of about 1.3 eq.

Generally, the strong acid may be any suitable strong acid known in the art with a stronger acidity than hydrogen bromide. In some embodiments, the strong acid is selected from sulfuric acid, hydrogen bromide, acids stronger than hydrogen bromide, and combinations thereof. In some embodiments, the strong acid is sulfuric acid.

In some embodiments, the base is a washing solution. In some embodiments, the base is selected from sodium hydroxide, potassium hydroxide, sodium carbonate, sodium bicarbonate, potassium carbonate, potassium bicarbonate and combinations thereof. In some embodiments, the base is sodium hydroxide.

In some embodiments, the method step of adding a base to the mixture comprises adding a base to the mixture through dropwise addition.

In some embodiments, at least one method step further comprises stirring the mixture.

Generally, cooling the reaction mixture during addition of the bromination agent and the strong acid benefits the reaction. Loss in yield and high impurities is observed at relatively high temperatures. In some embodiments, at least one method step further comprises cooling the mixture to a temperature less than about 5° C.

Generally, cooling the reaction mixture during the optional washing step of adding a base to the mixture benefits to reduce side reactions. In some embodiments, at least one method step further comprises cooling the mixture to a temperature less than about 10° C.

In some embodiments, at least one method step further comprises cooling the mixture to a temperature in a range of from about 0° C. to about 5° C.

In some embodiments, at least one method step further comprises heating the mixture to a temperature in a range of from about 8° C. to about 12° C.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Bromination Quantified by Equivalents

1.0 eq. ethyl 1-(3-chloropyridin-2-yl)-3-((phenylsulfonyl)oxy)-4,5-dihydro-1H-pyrazole-5-carboxylate in dichloromethane is cooled in a reactor to about 0-5° C. and about 1.0 eq. acetic acid and about 0.02 eq. PBr₃ are fed to the reactor. Next, 1.3 eq. hydrogen bromide gas is introduced into the reactor at about 0-5° C. Then 0.95 eq. sulfuric acid is fed into the reactor. The reaction mixture in the reactor is heated and held for 4 hours at about 8-12° C. After the reaction is completed, the reaction mass is washed by dropwise addition of 3.6 eq. of 10 wt % NaOH aqueous solution into the reaction mixture with agitation at less than 10° C., followed by phase separation at 20-25° C. to remove the aqueous phase. Finally, a second washing is performed by charging the organic phase to 0.4 eq. of 2 wt % NaOH solution at 0-10° C. to yield pH greater than 12. The phases are separated to yield ethyl 3-bromo-1-(3-chloropyridin-2-yl)-4,5-dihydro-1H-pyrazole-5-carboxylate solution in a yield of about 91.6%.

### Example 2. Small Scale Bromination

400 g ethyl 1-(3-chloropyridin-2-yl)-3-((phenylsulfonyl)oxy)-4,5-dihydro-1H-pyrazole-5-carboxylate in dichloromethane is cooled in a reactor to about 0-5° C. and 14.6 g acetic acid and 1.3 g PBr₃ are fed to the reactor. Next, 25.2 g hydrogen bromide is introduced into the reactor at about 0-5° C. Then 22.6 g sulfuric acid is fed into the reactor. The reaction mixture in the reactor is heated and held for 4 hours at about 8-12° C. After the reaction is completed, the reaction mass is washed two times by using 10% and 2% NaOH aqueous solutions. Typical yield of ethyl 3-bromo-1-(3-chloropyridin-2-yl)-4,5-dihydro-1H-pyrazole-5-carboxylate is about 90.15%.

### Example 3. Large Scale Bromination

200 kg ethyl 1-(3-chloropyridin-2-yl)-3-((phenylsulfonyl)oxy)-4,5-dihydro-1H-pyrazole-5-carboxylate in dichloromethane is cooled in a reactor to about 0-5° C. and 7.1 kg acetic acid and 0.64 kg PBr₃ are fed to the reactor. Next, 12.6 kg hydrogen bromide is introduced into the reactor at about 0-5° C. Then 11.3 kg sulfuric acid is fed into the reactor. The reaction mixture in the reactor is heated and held for 4 hours at about 8-12° C. After the reaction is completed, the reaction mass is washed two times by using 10% and 2% NaOH aqueous solutions. Typical yield of ethyl 3-bromo-1-(3-chloropyridin-2-yl)-4,5-dihydro-1H-pyrazole-5-carboxylate is about 91.6%.

Compared to the conventional process, the process according to the present disclosure demonstrates comparable yield with much less waste, much less bromination agent, and much less solvent.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “alkyl”, used either alone or in compound words such as “alkylthio” or “haloalkyl” includes straight-chain or branched alkyl, such as methyl, ethyl, npropyl, ipropyl, or the different butyl, pentyl or hexyl isomers.

The term “alkenyl” can include straight-chain or branched alkenes such as 1propenyl, 2propenyl, and the different butenyl, pentenyl and hexenyl isomers. “Alkenyl” also includes polyenes such as 1,2propadienyl and 2,4hexadienyl.

The term “alkynyl” includes straight-chain or branched alkynes such as 1propynyl, 2propynyl and the different butynyl, pentynyl and hexynyl isomers. “Alkynyl” can also include moieties comprised of multiple triple bonds such as 2,5hexadiynyl.

The term “alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkylthio” includes branched or straightchain alkylthio moieties such as methylthio, ethylthio, and the different propylthio, butylthio, pentylthio and hexylthio isomers.

The term “cycloalkyl” includes, for example, cyclopropyl, cyclobutyl, cyclopentyl and cyclohexyl. “Cycloalkylalkyl” indicates an alkyl group substituted with a cycloalky group and includes, for example, cyclopropylmethyl, cyclobutylethyl, cyclopentylpropyl and cyclohexylmethyl.

The term “cycloalkylamino” means the amino nitrogen atom is attached to a cycloalkyl radical and a hydrogen atom and includes groups such as cyclopropylamino, cyclobutylamino, cyclopentylamino and cyclohexylamino. “(Alkyl)cycloalkylamino” means a cycloalkylamino group where the hydrogen atom is replaced by an alkyl radical; examples include groups such as (alkyl)cyclopropylamino, (alkyl)cyclobutylamino, (alkyl)cyclopentylamino and (alkyl)cyclohexylamino.

The term “aryl” refers to an aromatic ring or ring system or a heteroaromatic ring or ring system, each ring or ring system optionally substituted. The term “aromatic ring system” denotes fully unsaturated carbocycles and heterocycles in which at least one ring of a polycyclic ring system is aromatic. Aromatic indicates that each of ring atoms is essentially in the same plane and has a p-orbital perpendicular to the ring plane, and in which (4n+2) π electrons, when n is 0 or a positive integer, are associated with the ring to comply with Hückel's rule. The term “aromatic carbocyclic ring system” includes fully aromatic carbocycles and carbocycles in which at least one ring of a polycyclic ring system is aromatic (e.g. phenyl and naphthyl). The term “heteroaromatic ring or ring system” includes fully aromatic heterocycles and heterocycles in which at least one ring of a polycyclic ring system is aromatic and in which at least one ring atom is not carbon and can contain 1 to 4 heteroatoms independently selected from the group consisting of nitrogen, oxygen and sulfur, provided that each heteroaromatic ring contains no more than 4 nitrogens, no more than 2 oxygens and no more than 2 sulfurs (where aromatic indicates that the Hückel rule is satisfied). The heterocyclic ring systems can be attached through any available carbon or nitrogen by replacement of a hydrogen on said carbon or nitrogen. More specifically, the term “aryl” refers to the moiety

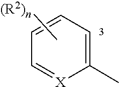

wherein R² and n are defined as above and the “3” indicates the 3-position for substituents on the moiety.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O.

The terms “alkylaminocarbonyl” and “dialkylaminocarbonyl” include, for example, CH₃NHC(═O), CH₃CH₂NHC(═O) and (CH₃)₂NC(═O).

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl. In the above recitations, when a compound of Formula (I) contains a heteroaromatic ring, all substituents are attached to this ring through any available carbon or nitrogen by replacement of a hydrogen on said carbon or nitrogen.

When a group contains a substituent which can be hydrogen, for example R⁴, then, when this substituent is taken as hydrogen, it is recognized that this is equivalent to said group being unsubstituted.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

What is claimed is:

1. A method of preparing a compound of Formula (II) 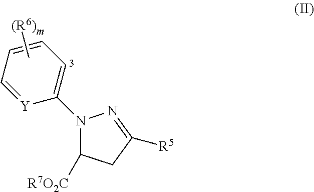
wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;
Y is N or CR⁸;
R⁸ is H or R⁶; and
m is 0, 1, 2, or 3, with the proviso that when X is CH then m is at least 1, the method comprising:
I) forming a mixture comprising
A) a compound of Formula (I) 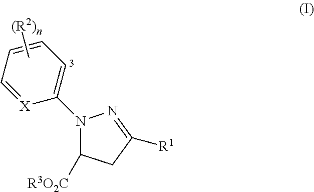
wherein R¹ is a sulfonate;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) a solvent; and
C) optionally a bromide;
II) introducing a bromination agent to the mixture;
III) introducing a strong acid to the mixture; and
IV) optionally adding a base to the mixture.

2. The method of claim 1, wherein m is 1, 2, or 3.

3. The method of any of claims 1-2, wherein R⁵ is Cl or Br.

4. The method of any of claims 1-3, wherein R⁶ is independently Cl or Br.

5. The method of any of claims 1-4, wherein one R⁶ is at the 3-position.

6. The method of any of claims 1-5, wherein R⁷ is C₁-C₄ alkyl.

7. The method of any of claims 1-6, wherein Y is N.

8. The method of any of claims 1-7, wherein the compound of Formula (II) is ethyl 3-bromo-1-(3-chloropyridin-2-yl)-4,5-dihydro-1H-pyrazole-5-carboxylate, having the following structure:

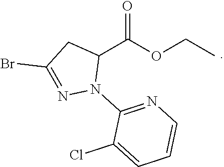 [SMILES: CCOC(=O)C1CC(Br)=NN1c1ncccc1Cl]

9. The method of any of claims 1-8, wherein n is 1, 2, or 3.

10. The method of any of claims 1-9, wherein R¹ is selected from methanesulfonate, benzenesulfonate, and p-toluenesulfonate.

11. The method of any of claims 1-10, wherein R² is independently Cl or Br.

12. The method of any of claims 1-11, wherein one R² is at the 3-position.

13. The method of any of claims 1-12, wherein R³ is C₁-C₄ alkyl.

14. The method of any of claims 1-13, wherein X is N.

15. The method of any of claims 1-14, wherein the compound of Formula (I) is ethyl 1-(3-chloropyridin-2-yl)-3-((phenylsulfonyl)oxy)-4,5-dihydro-1H-pyrazole-5-carboxylate, having the following structure:

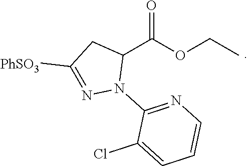

16. The method of any of claims 1-15, wherein the solvent is selected from dibromomethane, dichloromethane, acetic acid, ethyl acetate, acetonitrile, dichloroethane, dibromoethane, and combinations thereof.

17. The method of any of claims 1-16, wherein the solvent comprises acetic acid in an amount in a range of from about 0.1 eq. to about 10 eq., preferably about 0.5 eq. to about 6 eq.

18. The method of any of claims 1-17, wherein the bromide is selected from phosphorus oxybromide, phosphorus pentabromide, phosphorus tribromide, dibromo trialkyl phosphine and dibromo diphenyl phosphine, and combinations thereof.

19. The method of any of claims 1-18, wherein the bromination agent is selected from HBr, Br₂, and combinations thereof.

20. The method of any of claims 1-19, wherein the bromination agent is HBr.

21. The method of any of claims 1-20, wherein the strong acid is selected from sulfuric acid, hydrogen bromide, acids stronger than hydrogen bromide, and combinations thereof.

22. The method of any of claims 1-21, wherein the strong acid is sulfuric acid.

23. The method of any of claims 1-22, wherein the base is selected from sodium hydroxide, potassium hydroxide, sodium carbonate, sodium bicarbonate, potassium carbonate, potassium bicarbonate and combinations thereof.

24. The method of any of claims 1-23, wherein the base is sodium hydroxide.

25. The method of any of claims 1-24, wherein the method step of adding a base to the mixture comprises adding a base to the mixture through dropwise addition.

26. The method of any of claims 1-25, wherein at least one method step further comprises stirring the mixture.

27. The method of any of claims 1-26, wherein at least one method step further comprises cooling the mixture to a temperature in a range of from about 0° C. to about 5° C.

28. The method of any of claims 1-27, wherein at least one method step further comprises heating the mixture to a temperature in a range of from about 8° C. to about 12° C.
